(19) United States
(12) Patent Application Publication
(10) Pub. No.: US 2009/0070266 A1
(43) Pub. Date: Mar. 12, 2009

(54) SYSTEM AND METHOD FOR PHYSIOLOGICAL DATA AUTHENTICATION AND BUNDLING WITH DELAYED BINDING OF INDIVIDUAL IDENTIFICATION

(76) Inventors: Rahul C. Shah, San Francisco, CA (US); Mark D. Yarvis, Portland, OR (US)

Correspondence Address: Molly A. McCall;Intel Corporation, Intel Corporation c/o Intellevate, LLC, P.O. Box 52050, Minneapolis, MN, 55402, US

(21) Appl. No.: 11/851,530

(22) Filed: Sep. 7, 2007

(51) Int. Cl. H04K 1/00; A61B 5/00

(52) U.S. Cl. 705/51; 600/301

(57) ABSTRACT

A system and method for physiological data authentication and bundling with delayed binding of individual identification. In embodiments, the invention utilizes biometric data within a physiological data stream to allow for the late or delayed binding of the individual's identity to that data stream. In addition, the source of one or more additional data streams may be identified by cryptographically binding them to an original data stream. Other embodiments are described and claimed.

### BACKGROUND

A key characteristic of traditional data acquisition devices used in healthcare is anonymity. For example, a stethoscope, thermometer, or even an ECG device, typically does not know which patient is being measured. A key advantage of such traditional devices is that a patient's privacy is preserved.

Today, many healthcare applications involve a device that uses digital sensors to collect physiological data from one or more patients. The data collected is then stored in a server that may be used in the future to analyze the data. Since the data in the server is likely to belong to multiple patients, it is imperative to ensure that each piece of stored data is linked or bound to the correct patient. Thus, for a given piece or stream of sensed data, one must accurately identify the corresponding patient to ensure that it is accurately filed into the correct patient record in the server or displayed on the correct screen (typically near the patient).

Typical solutions used today to bind the identity of a patient to his or her digital physiological data compromises the privacy of the patient. For example, one solution involves the patient or healthcare professional to identify the patient to the device prior to physiological data being collected. This identification process may involve one or more of entering the patient's name into the device, swiping an identification card into the device, and/or supplying the device with a unique identifier and password. These approaches are cumbersome and error prone for numerous reasons. In addition, since the patient's identity is bound to his or her physiological data in the device, the patient's privacy may be at risk if the device is lost or compromised in some way.

### BRIEF DESCRIPTION OF THE DRAWINGS

FIG. 1 illustrates one embodiment of a process for physiological data authentication and bundling with delayed binding of individual identification.

FIG. 2 illustrates one embodiment of a system for physiological data authentication and bundling with delayed binding of individual identification.

FIG. 3 illustrates one embodiment of a logic flow for physiological data authentication and bundling with delayed binding of individual identification.

### DETAILED DESCRIPTION

Various embodiments of the present invention may be generally directed to a system and method for physiological data authentication and bundling with delayed binding of individual identification. In embodiments, the invention utilizes biometric data within a physiological data stream to allow for the late or delayed binding of the individual's identity to that data stream. In addition, the source of one or more additional data streams may be identified by cryptographically binding them to an original data stream. Other embodiments may be described and claimed.

Various embodiments may comprise one or more elements or components. An element may comprise any structure arranged to perform certain operations. Each element may be implemented as hardware, software, or any combination thereof, as desired for a given set of design parameters and/or performance constraints. Although an embodiment may be described with a limited number of elements in a certain topology by way of example, the embodiment may include more or less elements in alternate topologies as desired for a given implementation. It is worthy to note that any reference to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

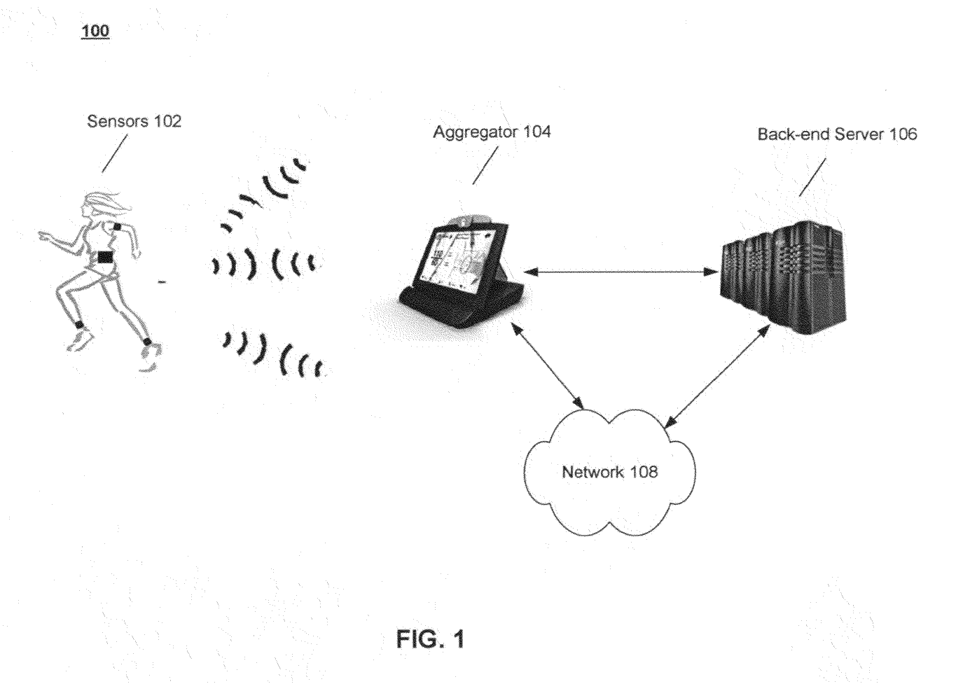

FIG. 1 illustrates one embodiment of a high level process 100 for physiological data authentication and bundling with delayed binding of individual identification. In one embodiment, process 100 comprises one or more sensors 102, a device or aggregator 104, a back-end server 106 and a network 108. At a high level and in an embodiment, real-time physiological data are collected for an individual via sensor(s) 102. It is possible to authenticate an individual via physiological sensor data such as, but not limited to, electrocardiograph (EKG/ECG), photoplethysmography (PPG) or phonocardiogram (PCG). Here, biometric data to identify the individual may be derived from a subset of the collected physiological data. It is important to note that only the collected physiological data, and not the user's explicit identity, are transmitted to aggregator 104. Thus, without having stored biometric data at aggregator 104 for the individual to compare against the collected physiological data, there is no way to identify the individual at aggregator 104.

Aggregator 104 then bundles the physiological data from the sensors and signs the bundled data, attesting that the bundled data streams belong to the same individual. Aggregator 104 transmits the bundled data to back-end server 106. The bundled data may be transmitted via network 108 (e.g., the Internet, a local area network (LAN), a wide area network (WAN), etc.) or via a direct connection between aggregator 104 and back-end server 106. All data in process 100 may be communicated via a wireless connection, a wired connection, or some combination of both.

Back-end server 106 validates the signed bundle. Biometric data derived from the physiological data in one or more data streams in the signed bundle is compared to previously obtained biometric data stored at the server 106 to identify the individual or patient to which it belongs. Only at this point is the identity of the individual bound to his or her physiological data. Each of the components or elements of process 100 will be discussed next in more detail.

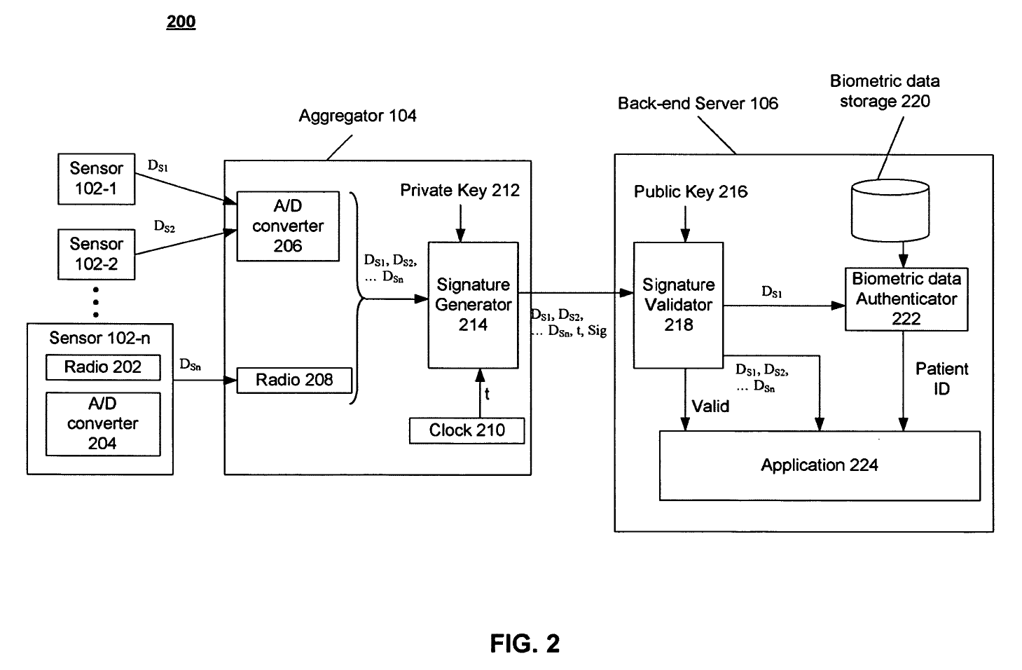

FIG. 2 illustrates one embodiment of a more detailed system 200 for the invention. The functionality of system 200 may be performed by more or less components than are illustrated in FIG. 2.

Referring to FIG. 2, system 200 includes one or more sensors 102 (102-1 through 102-n, where n is any positive integer). Real-time physiological data may be continuously collected for an individual via sensors 102. Real-time physiological data may also be collected at certain predetermined time intervals or on demand, for example. Sensors 102 may also be adapted to store real-time data via integrated long term storage, such as flash memory for example, and then to transmit the data to aggregator 104 at a later time. The integrated long term storage helps to ensure that no collected data are lost if there is no connection currently available with aggregator 104.

One or more of sensors 102 may be connected directly to aggregator 104. Here, an AID conversion of the collected data may be accomplished via an A/D converter 206 in aggregator 104. The collected data may also be wirelessly transmitted to aggregator 104 via, for example, Bluetooth technology, Zigbee technology or a proprietary system. In an embodiment, the A/D conversion of the collected data may be accomplished via an A/D converter in the sensor (such as A/D converter 204 in sensor 102-n). In an embodiment, the converted data may be transferred via a radio in a sensor (such as radio 202 in sensor 102-n) to radio 208 in aggregator 104. The invention is not limited to these example wireless technologies/examples. Alternatively, sensors 102 may transmit data to aggregator 104 via a wired connection, or some combination of wireless and wired connection technologies.

In an embodiment of the invention, sensors 102 may be small form factor devices that are worn by the individual and that are capable of monitoring and/or measuring physiological data or another type of data. Sensors 102, for example, may include an ECG device to measure a broad array of cardiovascular characteristics (e.g., heart rate variability, ECG amplitude, ST segment analysis, QT interval, etc.); a pulse oximeter unit to measure oxygenation level; a multiaxial accelerometer to measure activity level and orientation; a temperature sensor to measure temperature level; a unit to measure galvanic skin response; a pulse wave velocity monitor to monitor blood pressure; a minimally invasive or noninvasive glucometry monitor unit to measure blood sugar; and so forth. One or more of these sensors or units may be used either individually or in combination to collect physiological data for an individual. These examples are not meant to limit the invention. In fact, the invention contemplates the use of any means to monitor an individual.

As discussed above, aggregator 104 receives real-time (or stored) physiological data via sensors 102. As shown in FIG. 2, the physiological data or signals are represented as D_{S1}, D_{S2}, . . . , D_{Sn}.

Aggregator 104 bundles the received physiological data from a given acquisition. In embodiments, aggregator 104 has previously been configured as a device trusted by back-end server 106 and thus uses a private key 212 and a signature generator 214 to digitally sign and/or encrypt the bundled data transmitted to back-end server 106. Back-end server 106 has a corresponding public key 216, as shown in FIG. 2, to validate the signed bundle received from aggregator 104. In other embodiments, symmetric key cryptography may be used where both aggregator 104 and back-end server 106 will have access to the same secret key. Here, multiple streams of data may be cryptographically bound together, all of which belong to the same individual.

A clock 210 may also be used by aggregator 104 to generate and include a real or virtual timestamp, illustrated as t in FIG. 2. The timestamp may be included in the signed data bundle to prevent replay. The resulting signed data bundle may be represented as (D_{S1}, D_{S2}, . . . , D_{Sn}, t, Sig), as illustrated in FIG. 2. This signed data bundle allows the trusted source (i.e., aggregator 104) to attest that the data originated from the same individual, however, the exact identity of the individual is not known or specified by aggregator 104.

In one embodiment, aggregator 104 may be any device capable of performing the functionality of the invention described herein. Aggregator 104 may be implemented as part of a wired communication system, a wireless communication system, or a combination of both. In one embodiment, for example, aggregator 104 may be implemented as a mobile computing device having wireless capabilities. A mobile computing device may refer to any device having a processing system and a mobile power source or supply, such as one or more batteries, for example.

Examples of embodiments of a mobile computing device that may be adapted to include the functionality of the present invention include a laptop computer, ultra-mobile computer, portable computer, handheld computer, palmtop computer, personal digital assistant (PDA), cellular telephone, combination cellular telephone/PDA, smart phone, pager, one-way pager, two-way pager, messaging device, data communication device, and so forth.

Examples of such a mobile computing device also may include computers that are arranged to be worn by a person, such as a wrist computer, finger computer, ring computer, eyeglass computer, belt-clip computer, arm-band computer, shoe computers, clothing computers, and other wearable computers.

As described above, the signed data bundle represented as (D_{S1}, D_{S2}, . . . , D_{Sn}, t, Sig) is received at back-end server 106. A signature validator 218 uses public key 216 to validate the timestamp and digital signature in the data bundle. If the input is valid, back-end server 106 knows that the data bundle originated from a trusted device (i.e., aggregator 104) and that the data in the bundle came from a single individual. Signature validator 218 sends a valid signal to an application 224, along with the data streams D_{S1}, D_{S2}, . . . , and D_{Sn}.

In an embodiment, one or more of the streams of data in the data bundle are used to identify the user at back-end server 106 via biometric authentication. In FIG. 2, D_{S1} represents the data stream that is used to identify the user. Signature validator 218 forwards D_{S1} to a biometric data authenticator 222. Authenticator 222 uses D_{S1} and a biometric data storage 220 to determine the identity of the individual. For example, assume that back-end server 106 is located at a hospital. Here, biometric data storage 220 may store a biometric sample from each of its patients. D_{S1} is compared to the stored biometric samples to determine a match, and thus the identity of the patient. Note that without such a biometric data storage 220, it is not possible to determine the identify of the patient as this is the only part of system 200 that stores the patient's identity. Biometric data authenticator 222 forwards the patient's identification to application 224. Application 224 binds the identity of the patient to his or her streams of data.

In various embodiments, system 200 may be implemented as a wireless system, a wired system, or a combination of both. When implemented as a wireless system, system 200 may include components and interfaces suitable for communicating over a wireless shared media, such as one or more antennas, transmitters, receivers, transceivers, amplifiers, filters, control logic, and so forth. An example of wireless shared media may include portions of a wireless spectrum, such as the RF spectrum and so forth. When implemented as a wired system, system 200 may include components and interfaces suitable for communicating over wired communications media, such as input/output (I/O) adapters, physical connectors to connect the I/O adapter with a corresponding wired communications medium, a network interface card (NIC), disc controller, video controller, audio controller, and so forth. Examples of wired communications media may include a wire, cable, metal leads, printed circuit board (PCB), backplane, switch fabric, semiconductor material, twisted-pair wire, co-axial cable, fiber optics, and so forth.

Operations for the above embodiments may be further described with reference to the following figures and accompanying examples. Some of the figures may include a logic flow. Although such figures presented herein may include a particular logic flow, it can be appreciated that the logic flow merely provides an example of how the general functionality as described herein can be implemented. Further, the given logic flow does not necessarily have to be executed in the order presented unless otherwise indicated. In addition, the given logic flow may be implemented by a hardware element, a software element executed by a processor, or any combination thereof.

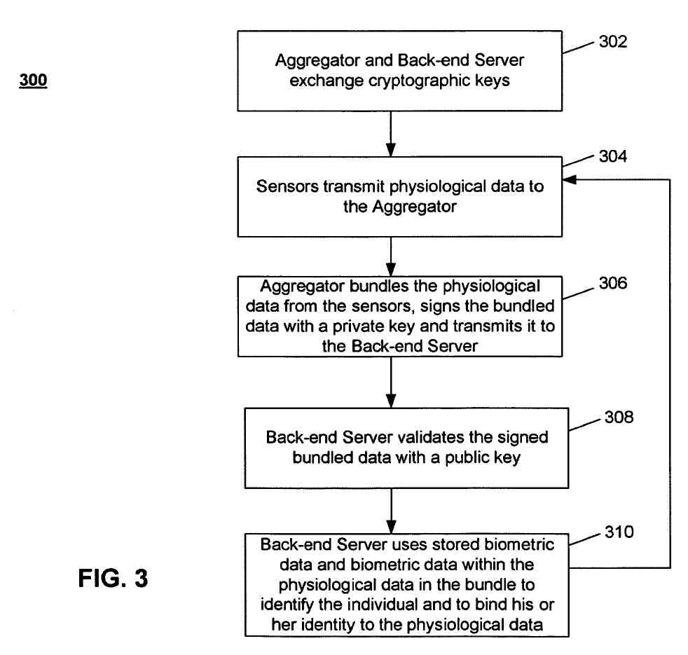

FIG. 3 illustrates one embodiment of a logic flow 300. The logic flow 300 may be representative of the operations executed by one or more embodiments described herein, for example, the operations executed by system 200.

Referring to FIG. 3, an aggregator and a back-end server (such as aggregator 104 and back-end server 106) exchange cryptographic keys (block 302). One or more sensors (such as sensors 102) send physiological data or signals to the aggregator (block 304). The aggregator bundles the physiological data and signs the bundled data with a private key. The signed data bundle is transmitted to the back-end server (block 306). The back-end server validates the signed data bundle with its public key (block 308). The back-end server then uses stored biometric data and biometric data derived from the signed data bundle to identify the individual. An application binds the identity of the individual to the data streams (block 310).

Various embodiments may be implemented using hardware elements, software elements, or a combination of both. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Some embodiments may be implemented, for example, using a machine-readable or computer-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

Numerous specific details have been set forth herein to provide a thorough understanding of the embodiments. It will be understood by those skilled in the art, however, that the embodiments may be practiced without these specific details. In other instances, well-known operations, components and circuits have not been described in detail so as not to obscure the embodiments. It can be appreciated that the specific structural and functional details disclosed herein may be representative and do not necessarily limit the scope of the embodiments.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A system, comprising:
a device to receive one or more streams of physiological data measured from an individual, wherein the device to aggregate the one or more received streams of physiological data into a data bundle and to sign the data bundle; and
a back-end server to receive the signed data bundle from the device, wherein the back-end server to validate the signed data bundle and, if valid, to determine an identity for the individual from the signed data bundle and to bind the identity of the individual to the one or more streams of physiological data.

2. The system of claim 1, wherein the signed data bundle to include a timestamp.

3. The system of claim 1, wherein the identity of the individual is determined by comparing previously stored biometric data for the individual and biometric data derived from the signed data bundle.

4. The system of claim 3, wherein the previously stored biometric data is stored at the back-end server.

5. The system of claim 1, wherein the one or more streams of physiological data are cryptographically bound together in the signed data bundle.

6. The system of claim 1, wherein the device uses a private key to sign the data bundle and wherein the back-end server to use a public key corresponding to the private key to validate the signed data bundle.

7. The system of claim 1, wherein the device uses a symmetric key to sign the data bundle and wherein the back-end server uses the symmetric key to validate the signed data bundle.

8. A method, comprising:
aggregating one or more received streams of physiological data into a data bundle;
signing the data bundle;
validating the signed data bundle at a back-end server;
if valid, determining an identity for the individual from the signed data bundle at the back-end server; and
binding the identity of the individual to the one or more streams of physiological data at the back-end server.

9. The method of claim 8, wherein the signed data bundle to include a timestamp.

10. The method of claim 8, wherein the determining the identity of the individual comprises:
comparing previously stored biometric data for the individual and biometric data derived from the signed data bundle for a match.

11. The method of claim 10, wherein the previously stored biometric data is stored at the back-end server.

12. The method of claim 8, wherein the one or more streams of physiological data are cryptographically bound together in the signed data bundle.

13. The method of claim 8, further comprising:
using a private key to sign the data bundle; and
and using a public key corresponding to the private key to validate the signed data bundle.

14. The method of claim 8, further comprising:
using a symmetric key to sign the data bundle; and
and using the symmetric key to validate the signed data bundle.

15. A machine-readable medium containing instructions which, when executed by a processing system, cause the processing system to perform a method, the method comprising:
aggregating one or more received streams of physiological data into a data bundle;
signing the data bundle;
validating the signed data bundle at a back-end server;
if valid, determining an identity for the individual from the signed data bundle at the back-end server; and
binding the identity of the individual to the one or more streams of physiological data at the back-end server.

16. The machine-readable medium of claim 15, wherein the signed data bundle to include a timestamp.

17. The machine-readable medium of claim 15, wherein the determining the identity of the individual comprises:
comparing previously stored biometric data for the individual and biometric data derived from the signed data bundle for a match.

18. The machine-readable medium of claim 17, wherein the previously stored biometric data is stored at the back-end server.

19. The machine-readable medium of claim 15, wherein the one or more streams of physiological data are cryptographically bound together in the signed data bundle.

20. The machine-readable medium of claim 15, further comprising:
using a private key to sign the data bundle; and
and using a public key corresponding to the private key to validate the signed data bundle.

21. The machine-readable medium of claim 15, further comprising:
using a symmetric key to sign the data bundle; and
and using the symmetric key to validate the signed data bundle.
